(19) United States
(12) Patent Application Publication
(10) Pub. No.: US 2007/0255290 A1
(43) Pub. Date: Nov. 1, 2007

(54) Tonsillith removing device

(76) Inventor: Hongjun Pan, Belmont, MA (US)

Correspondence Address: Hongjun Pan, 111 Hammond Road, Belmont, MA, 02478, US

(21) Appl. No.: 11/412,727

(22) Filed: Apr. 28, 2006

(51) Int. Cl. A61B 17/22

(52) U.S. Cl. 606/127; 606/110

(57) ABSTRACT

A tonsillith removing device for use in removing tonsillith and other undesired matter from a user's tonsil crypts is described. The device comprises a suction channel element and a suction mechanism unit. The suction force is provided by an electric fan installed in the suction mechanism unit housing. The device has a retention feature for retaining the sucked tonsillith and undesired matter. The suction channel element can be easily detached from the suction mechanism unit and the retained tonsillith and undesired matter can be cleaned.

### BACKGROUND OF THE INVENTION

1. Field of the Invention

The present invention relates to a device for personal cleaning and, more particularly, to a device for removing the undesired matter from user's tonsils and, even more particularly, the invention relates to a device for removing tonsillith (or called tonsil stone, tonsillolith, calculi of the tonsil) from the tonsil crypts (or called tonsil pockets).

2. Background Information

About 85% of people suffer the halitosis (bad breath) which can be chronic or pathological disorder stemming from non-oral sources as well as oral sources. One of the main oral sources is the tonsillith formed in the tonsil crypts. There are more than 5% of people from young kids to elder adults having halitosis caused by tonsillith. The tonsillith is the accumulation of food particles, bacteria, leukocyte, dead white cells, and dried mucus. The accumulation products are colored white or yellow, have a rough texture similar to cottage cheese or cauliflower, contain high quantities of sulfa and convey an extremely foul odor which causes the halitosis (bad breath). Such bad breath severely affects patients' social activities and romantic relationship. Patients use Q-tips to push the tonsil crypts and squeeze the tonsillith out or use a finger to reach the tonsillith and scrape it out of the crypts. Both the methods are very inefficient and frequently hurt the tonsil. The tonsillith will come up again in about one to two weeks or longer depending on each person. Brushing teeth and tongue or gargling mouth with mouthwash can temporarily reduce the bad breath, but such bad breath will be back to original level in about 2 hours if the source of the cause is not removed. A more drastic method, a tonsillectomy, may be used to remove the tonsil. Patients suffer through the surgery. Moreover, the tonsil is an useful organ and should not be removed if there is no threat to life. To the extensive search by the inventor, however, there is no prior art with the same field of the invention as of the present invention. Therefore, it is strongly desirable to have an efficient and easily-used device available for the patients to remove the tonsillith, they can restore their fresh breath, have normal social activities and enhance the romantic relationship, the present invention will provide a solution for those patients.

### SUMMARY OF THE INVENTION

It is therefore one objective of the present invention to provide a tonsillith removing device that allows the tonsillith being easily removed by the device.

It is still another objective of the present invention to provide a tonsillith removing device that has a round bottle cap shaped suction channel element with an angled tube extended from the center.

It is still another objective of the present invention to provide a tonsillith removing device that has a cylinder-like suction mechanism unit to which the angled suction channel element is connected by threads. The suction mechanism unit may be electrically powered.

It is still another objective of the present invention to provide a tonsillith removing device that the housing of the suction mechanism unit can be used as a handle to allow the user easily operating the device.

It is a further objective of the present invention to provide a tonsillith removing device that incorporates a tonsillith retention feature to retain the tonsilliths removed from the tonsil crypts.

### BRIEF DESCRIPTION OF THE DRAWINGS

FIG. 1 is the cross-sectional view of the tonsillith removing device.

FIG. 2 shows the angled side view of the tonsillith removing device according to the present invention.

FIG. 3 is the partial cross-sectional view of the tonsillith removing device placed in a tonsil crypt with a tonsillith in it.

FIG. 4 is the partial cross-sectional view of the tonsillith removing device sucking out the tonsillith from the tonsil crypt.

### DETAILED DESCRIPTION OF THE PREFERRED EMBODIMENTS

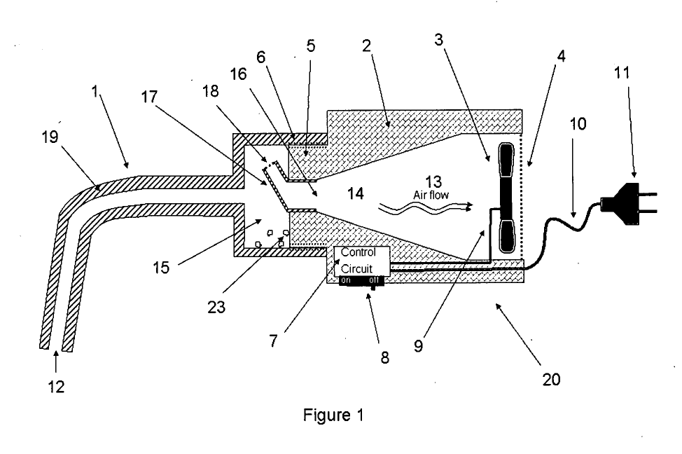

Referring in detail to the FIG. 1, the tonsillith removing device 20 comprises the suction channel element 1 and the suction mechanism unit 2. The suction channel element 1 has a round bottle cap shaped portion with threads 6 inside and a tube 19 is extended from the center of the round bottle cap shaped portion. One opening 12 of the tube has a smooth edge and surface that is placed on the tonsil crypts when it is in use. The tube can be angled or straight, however, the angled tube allows user to have a better view of the location of tonsillith in the back of the mouth during the operation. The suction mechanism unit 2 has a round cylinder shape, a short section 5 of the cylinder shape has smaller size with threads 6 on the out side surface. The suction mechanism unit 2 has two open ends with different sizes. A electric fan 3 is installed at the large open end. The size of inner chamber 14 gradually decreases towards to the smaller open end 16. An angled tube 17 is inserted to the open end 16. A grate material 18 is attached to the open end of the angled tube 17 to prevent the sucked tonsilliths 23 from moving into the chamber 14. A grate material 4 is attached to the large open end of the suction mechanism unit 2 to allow air flowing 13 out. A control circuit 7 and a switch 8 are installed in the suction mechanism unit 2, the fan 3 is connected to the control circuit 7 with insulated wires 9, and an electric power cord 10 with a plug 11 is connected to the control circuit 7. The suction channel element 1 is connected to the suction mechanism unit 2 with the treads 6. The space 15 is the retention area to retain the tonsilliths and undesired matter sucked from the tonsil crypts.

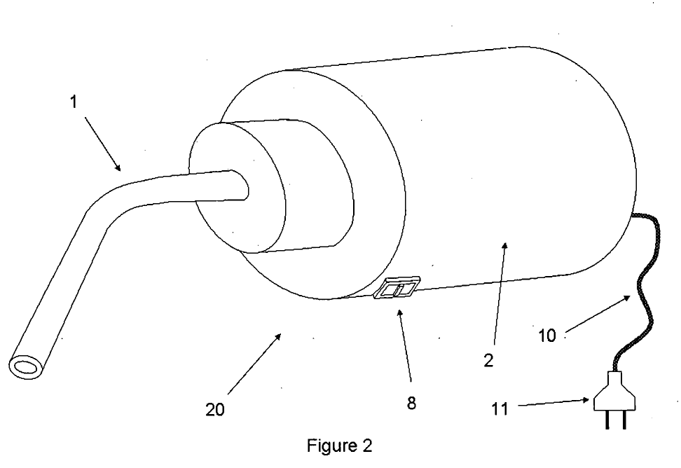

FIG. 2 shows the angled side view of the tonsillith removing device according to the present invention. The user can hold the suction mechanism unit housing 2 as a handle to operate the tonsillith removing device, the switch 8 is easily reached by the user's finger to control the operation.

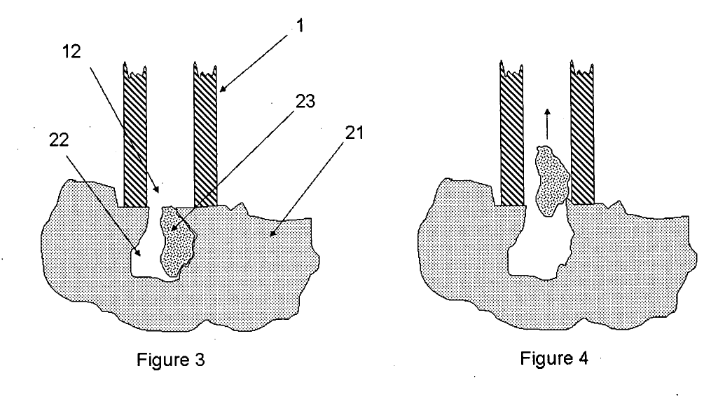

The usage of the tonsillith removing device is illustrated in FIGS. 3 and 4. When the tonsillith develops into a certain size in the tonsil crypts and can be seen, the user can use the present invention to remove the tonsillith. To use the present tonsillith removing device, the user plugs the electric plug 11 into an electric outlet, faces to a mirror with enough light to see the location of the tonsillith in the back of the mouth, holds the suction mechanism unit housing and gently place the open end 12 of the suction channel element 1 on the tonsil crypt 22 location where the tonsillith 23 resides shown in FIG. 3, switches on the power by a finger, the fan 3 turns and blows the air out of the suction mechanism unit housing chamber 14 and generates the suction force in the suction channel element 1 to pull the tonsillith 23 out of the crypt 22 shown in FIG. 4. Therefore the operation is easy and efficient. After all visible tonsilliths are removed, the suction channel element 1 can be detached from the suction mechanism unit 2 and the tonsilliths 23 retained in the space 15 of the device can be removed, the suction channel element 1 and the suction mechanism unit 2 can be cleaned for next use.

While the preferred embodiments of the present invention have been illustrated and described, it will be obvious to those skilled that various changes and modification may be made therein without departing from the scope of the present invention as defined in the claims.

The invention claimed is:

1. A tonsillith removing device for use in removing undesired matter from a user's tonsil crypts. The tonsillith removing device comprises: a suction channel element and a suction mechanism unit.

2. The tonsillith removing device of claim 1, wherein the suction channel element has a round bottle cap shape with threads inside, an open tube extends from the center of the bottle cap portion. The tube can be angled or straight. The end of the tube has smooth edge and surface and is placed at the tonsil crypts when it is in use.

3. The tonsillith removing device of claim 1, wherein the suction mechanism unit has a cylinder shape with two different size open ends, a section of the cylinder shape has smaller size with threads at outside surface. An electric fan is installed inside the suction mechanism unit housing at the large open end to provide sucking force. The size of the suction mechanism unit housing gradually decreases towards the smaller open end that is at the smaller cylinder side. An angled tube is inserted into the smaller open end. A grate material is attached to the end surface of the angled tube. The fan is electrically powered and controlled by a switch and a control circuit. A grate material is attached to the large open end of the suction mechanism unit. An electric power cord with plug is connected to the control circuit.

4. The tonsillith removing device of claim 1, wherein the suction channel element is mounted on the suction mechanism unit by threads. There is hollow space inside between the end surface of the suction mechanism unit and the suction channel element, such hollow space has retention function to retain the sucked tonsilliths and undesired matter from the tonsil crypts.
